(19) United States
(12) Patent Application Publication
(10) Pub. No.: US 2010/0076204 A1
(43) Pub. Date: Mar. 25, 2010

(54) PROCESS FOR THE PREPARATION OF LEVETIRACETAM

(75) Inventors: Massimiliano FORCATO, Galzignano Terme (IT); Ivan MICHIELETTO, Venezia (IT); Paolo MARAGNI, Virgilio (IT); Franco MASSACCESI, Grancona (IT); Livius COTARCA, Cervignano del Friuli (IT)

Correspondence Address: LUCAS & MERCANTI, LLP, 475 PARK AVENUE SOUTH, 15TH FLOOR, NEW YORK, NY, 10016, US

(73) Assignee: ZACH SYSTEM S.P.A., Bresso (Milano) (IT)

(21) Appl. No.: 12/374,948

(22) PCT Filed: Jul. 20, 2007
(86) PCT No.: PCT/EP2007/057503

§ 371 Date: Feb. 18, 2009

(30) Foreign Application Priority Data
Jul. 25, 2006 (EP) 06012439.0
Jul. 20, 2007 (EP) PCT/EP2007/057503

(51) Int. Cl. C07D 207/12

(52) U.S. Cl. 548/550

(57) ABSTRACT

The present invention relates to a process for the preparation of levetiracetam and, more particularly, to an improved process for the preparation of levetiracetam characterized by a crystallization-induced dynamic resolution of a diastereoisomeric mixture of an (±)-alpha-ethyl-2-oxo-1-pyrrolidine acetamide derivative.

The present invention relates to a process for the preparation of levetiracetam and, more particularly, to an improved process for the preparation of levetiracetam characterized by a crystallization-induced dynamic resolution of a diastereoisomeric mixture of an (±)-alpha-ethyl-2-oxo-1-pyrrolidine acetamide derivative.

The invention also discloses novel intermediates and their use in the preparation of the enantiomerically pure end-product.

Levetiracetam, (−)-(S)-alpha-ethyl-2-oxo-1-pyrrolidineacetamide, is a drug useful as a protective agent for treating and preventing hypoxic and ischemic type aggressions of the central nervous system. It is the active ingredient of KEPPRA®, tablets and flavored liquid, indicated as adjunctive therapy in the treatment of partial onset seizures in adults and children four years of age and older with epilepsy.

Levetiracetam was first described in U.S. Pat. No. 4,837,223 (UCB Societe Anonyme) where it is stated that it has particular therapeutic properties compared to the known racemic form (non proprietary name etiracetam). The S-enantiomer, for example, has a ten times higher protective activity against hypoxia and a four times higher protective activity against cerebral ischemia than the racemic mixture

US '223 describes a method for the preparation of levetiracetam which comprises reacting (−)-(S)-alpha-ethyl-2-oxo-1-pyrrolidineacetic acid successively with alkylhaloformate and with ammonia. Said acid intermediate is, in turn, obtained from racemic (±)-alpha-ethyl-2-oxo-1-pyrrolidine acetic acid by a classic optical resolution according to known methods. In example 1 of the above US patent, ethyl (±)-alpha-ethyl-2-oxo-1-pyrrolidine acetate is hydrolyzed to give the corresponding racemic acid in the presence of sodium hydroxide; said acid is subjected to chemical resolution by reaction with an optically active base, (+)-(R)-(1-phenylethyl)-amine, selective crystallization of diastereoisomeric salts thereof and isolation of the desired enantiomeric form; finally, the resultant (−)-(S)-alpha-ethyl-2-oxo-1-pyrrolidineacetic acid is converted into the corresponding amide via activation of the carboxyl residue with ethyl chloroformate, in accordance with the following reaction scheme:

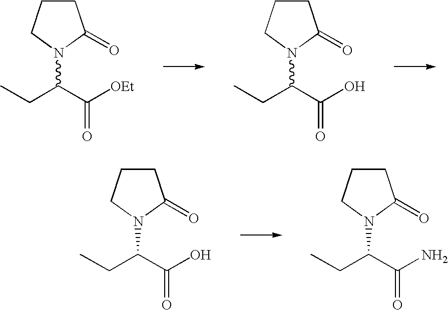

Several alternative processes for the preparation of levetiracetam have been disclosed in the art.

WO 03/014080 (UCB S.A.) describes an improved process for the preparation of levetiracetam and analogues thereof comprising the ammonolysis reaction of the corresponding ester derivatives in the presence of water.

U.S. Pat. No. 6,107,492 (Daicel Chem; UCB) and U.S. Pat. No. 6,124,473 (UCB) describe the preparation of levetiracetam by optical resolution of etiracetam by means of preparative high performance liquid chromatography or continuous simulated moving bed chromatographic system.

GB 2,225,322 (UCB) describes a process for the preparation of levetiracetam by hydrogenolysis of (S)-alpha-[2-(methylthio)-ethyl]-(2-oxo-1-pyrrolidine)-acetamide in the presence of a desulfurizing agent such as NaBH4/NiCl2 6H2O, nickel Raney W-2 or nickel Raney T-1.

WO 01/64637 (UCB Farchim) describes the preparation of levetiracetam by asymmetric hydrogenation of (Z) or (E)-2-(2-oxotetrahydro-1H-1-pyrrolyl)-2-butenamide by using a chiral catalyst.

EP 162,036 (UCB) describes the preparation of levetiracetam by reacting (S)-2-aminobutanamide with an alkyl 4-halobutyrate or with a 4-halobutyryl halide, and subsequent cyclization of alkyl (S)-4-[[1-(aminocarbonyl)-propyl]-amino-butyrate or of (S)-N-[1-(aminocarbonyl)-propyl]-4-halobutanamide thus obtained.

WO 2004/069796 (Teva Pharmaceutical Industries) describes a process for preparing levetiracetam which comprises reacting (S)-2-aminobutyrramide hydrochloride and 4-chlorobutyl chloride in a solvent selected from acetonitrile and methyl tertbutyl ether in the presence of a strong base and recovering the crude product.

US 2005/0182262 (Dr. Reddy's Laboratories) describes the preparation of (S)-2-aminobutyrramide hydrochloride, intermediate useful for the manufacture of levetiracetam via reaction with 4-chlorobutyl chloride.

WO 2004/076416 (Farma Lepori S.A.) describes a process to levetiracetam by means of deaminomethylation of a sufficiently pure enantiomer S-intermediate of formula

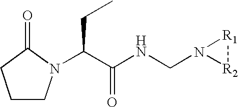

or a salt thereof

Said intermediate is obtained from the corresponding racemic mixture by reaction with an amine resolving agent and selective crystallization of a diastereoisomeric salt thereof.

In order to obtain the end-product in the correct stereochemical configuration, most processes for the preparation of levetiracetam require a supplementary step of optical resolution.

In accordance with US '223, (−)-(S)-alpha-ethyl-2-oxo-1-pyrrolidineacetamide can not be obtained directly from the racemic mixture by separating the desired enantiomer.

Thus, as underlined above, in US '223 the resolution step is carried out on the intermediate (±)-alpha-ethyl-2-oxo-1-pyrrolidineacetic acid.

Said procedure has an intrinsic drawback due to separation of the S-enantiomer from the corresponding racemic mixture by classic optical resolution which, necessarily, leads to a loss of 50% of the acid substrate used.

Processes disclosed in the art try to bypass the above problem of loss of yields in levetiracetam coming from the resolution process by using chiral substrates in asymmetric syntheses, enantioselective reductions, chromatographic separations or classical resolutions of specific intermediates which allows recycling the opposite undesired (R)-enantiomer.

Moreover, in the literature only a limited number of documents relating to a resolution process analogue to that object of the invention, are reported.

Hereinafter, we cite the most significant ones:

WO 2005/121117 (Sumitomo Chemical Company) describes a process for the production of optically active compounds (Ia) or (Ib) which comprises the first step of reacting a compound II with a compound III in the presence of a base to form a diastereomer mixture (I) and the second step of crystallizing an optically active compounds (Ia) or (Ib) from the mixture (I) while making the mixture (I) undergo equilibrium epimerization in the presence of a base; and a process for the production of optically active compounds (IVa) or (IVb) by utilizing the above process.

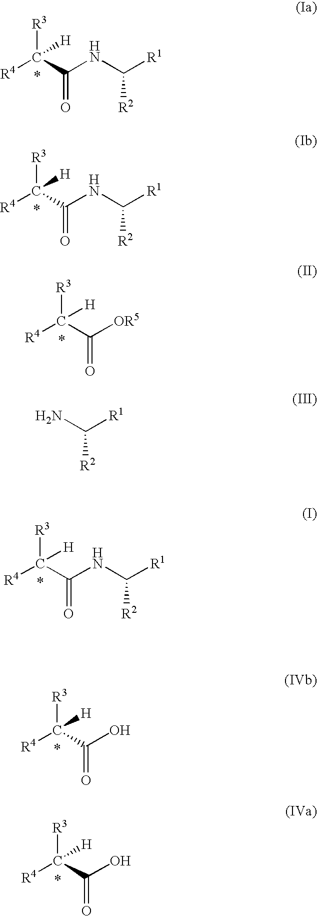

EP 0719755, in the name of the same Applicant, describes a process for the preparation of 2-(2-fluoro-4-biphenyl)-propionic acid enantiomers comprising a II order resolution of ketals of formula

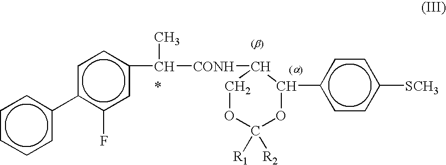

wherein R₁ and R₂ have the meanings reported in the description; the asterisk shows the chiral carbon atom and the asymmetric atoms marked by α and β have both R and S configuration.

Nevertheless, said processes are carried out by means of different optically active amines and/or resolution conditions.

Therefore, it would be desirable to provide new alternative processes for preparing levetiracetam on an industrial scale which are able to overcome problems related to separation of suitable optical isomer, in particular, by preventing loss of yield due to the resolution of key intermediates.

We have now surprisingly found an improved process for the preparation of levetiracetam by a method of dynamic resolution which does not show the drawbacks of the prior art and allows obtaining the desired enantiomer in good yields and with high purity starting from known raw materials.

Therefore, object of the present invention is a process for the preparation of levetiracetam which comprises a crystallization-induced dynamic resolution of a diastereoisomeric mixture of an (±)-alpha-ethyl-2-oxo-1-pyrrolidine acetic amide of formula

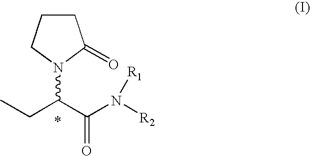

wherein

R₁ is hydrogen or a benzyl group;

R₂ is a 1-phenylethyl group optionally substituted on the phenyl ring by nitro or (C₁-C₄)-alkoxy; a 1-phenylpropyl group; a 1-naphtylethyl group; a 3-pinylmethyl group;

or R₁ and R₂ taken together form a 5 or 6 membered saturated heterocycle containing from 1 to 3 heteroatoms selected among nitrogen, oxygen and sulfur, substituted by one or more (C₁-C₄)-alkyl group;

from basic catalysis.

The acetic amides of formula I have one stereogenic centre in their structure being the carbon atom linked to the nitrogen atom of the pyrrolidine moiety. It is marked by an asterisk in formula I.

In addition, the compounds of formula I have at least a second stereogenic centre in the meanings of the residues R₁ and R₂.

Kinetic resolutions allow separation of stereoisomers from each other using differences in reaction rates of said stereoisomers with a substrate. In dynamic process (DKR) starting stereoisomers can interconvert and only one of them is able to react leading to situations where the product of separation has very high diastereoisomeric excess and very high yielding. Crystallization-induced dynamic resolution (CIDR, Andersson N. G., Org. Proc. Res. & Dev., 2005, 9, 800) refers to processes where the crystallization of one stereoisomer is the driving force of the dynamic process i.e. interconversion of stereoisomers.

The improved process object of the invention has the advantage of requiring no additional steps such as, for example, racemization of the opposite enantiomer and further resolution, in order to increase yield of product.

The process object of the invention provides a simple and readily industrialized alternative preparation of enantiomerically pure levetiracetam from an amide intermediate which is in turn easily obtained by conventional methods from substrate known in the art.

In fact, diastereoisomeric amides which may be used in the resolution process of the invention, are obtained in accordance with known methods by simply reacting substrates (±)-alpha-ethyl-2-oxo-1-pyrrolidine acetic acid or a derivatives thereof such as, for example, (C₁-C₄)-alkyl (±)-alpha-ethyl-2-oxo-1-pyrrolidine acetate, with a suitable optically active amine which is able to form a diastereoisomeric mixture.

According to the invention the amidation reaction is carried out with an amine of formula

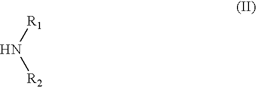

wherein residues R₁ and R₂ have the meanings defined in formula I; nevertheless, the skilled person will realize that alternative optically active amines may be use without departing from the spirit of the invention.

In the process object of the present invention, the optically active amines of formula II are, preferably, amines wherein residue R₁ is a hydrogen atom i.e. primary amines Between primary amines (+)-(R)-(1-phenylethyl)-amine, (−)-(S)-(1-phenylethyl)-amine, (+)-(R)-1-[(4-metoxyphenyl)-ethyl]-amine, (−)-(S)-1-[(4-metoxyphenyl)-ethyl]-amine, (+)-(R)-1-[(4-nitrophenyl)-ethyl]-amine, (−)-(S)-1-[(4-nitrophenyl)-ethyl]-amine, (+)-(R)-(1-phenylpropyl)-amine, (−)-(S)-(1-phenylpropyl)-amine, (+)-(R)-(1-naphtylethyl)-amine, (−)-(S)-(1-naphtylethyl)-amine, (+)-3-aminomethyl-pinane and (−)-3-aminomethylpinane are preferred.

Alternatively, amines of formula II wherein residue R₁ is different from hydrogen i.e. secondary amines may be used in the process. Examples of secondary amines of formula II are (R)-(+)-N-benzyl-(1-phenylethyl)-amine and (S)-(−)-N-benzyl-(1-phenylethyl)-amine or those wherein residues wherein R₁ and R₂ form a heterocyclic ring such as (−)-(R)-3-methyl-piperidine, (+)-(S)-3-methyl-piperidine, (−)-(R)-2-methyl-piperidine, (+)-(S)-2-methyl-piperidine, (−)-(R)-2-methylpyrrolidine, (+)-(S)-2-methylpyrrolidine, (2R,5S)-2,5-dimethyl-pyrrolidine and (2R,6R)-2,6-dimethylpiperidine. Nevertheless, the use of said secondary amines, although they are efficient in the dynamic resolution, may entail some problems during subsequent steps of the process for the preparation of levetiracetam

Particularly preferred amine is (+)-(R)-(1-phenylethyl)-amine, thus, dynamic resolution from basic catalysis is preferably carried out on the diastereoisomeric mixture of the compound (±)-(R,S)-alpha-ethyl-2-oxo-1-pyrrolidine-acet-N-(+)-(R)-(1-phenylethyl)-amide.

Substrate (±)-alpha-ethyl-2-oxo-1-pyrrolidine acetic acid may be prepared by saponifying the corresponding alkyl esters in the presence of a base according to the teachings disclosed in US '223.

While, in GB 1,309,692 the synthesis of said alkyl esters by condensation reaction between 2-oxo-pyrrolidine and haloalkyl carboxylate in the presence of strong base is described.

For example, the amidation reaction may be carried out by reacting racemic lower alkyl 2-oxopirrolidine butyrate with a suitable optically active amine in the presence of an inert solvent and a base.

It is evident to the man skilled in the art the advantage deriving from the use of the ester derivatives as reaction substrate. Use which allows to reduce synthetic steps disclosed in US '223.

According to the invention, said diastereoisomeric amide intermediate gives rise to a second order resolution process when subjected to basic catalysis conditions in the presence of suitable solvents or mixture thereof

Process object of the invention results in a highly efficient conversion of the diastereoisomeric mixture into the stereoisomer wherein chiral center in alpha position has the desired S-configuration. Moreover, said stereoisomer is easily isolated from the reaction mixture in good yields and high diastereoisomeric excess.

Dynamic resolution of the invention is carried out in the presence of a catalytic amount of a base, preferably, an organic base.

Preferably, an organic base such as 1,4-diazabicyclo[2.2.2]octane (DABCO), 1,8-diazabicyclo[5.4.0]undec-7-ene (DBU), 1,5,7-triazabicyclo[4.4.0]dec-5-ene (TBD) and alkali metal alkoxide is used.

More preferably, dynamic reaction is carried out in the presence of (C₁-C₄)-alkali metal alkoxide.

Still more preferably, the organic base is sodium methoxide.

The catalytic amount of base is preferably comprised between 5% and 15% with regard to the amide substrate.

Preferably, the catalytic amount of base is around 10%.

The reaction takes place in the presence of one or more inert organic solvents or mixture thereof.

Suitable organic solvents are aromatic or aliphatic hydrocarbons and aliphatic ethers.

Preferred organic solvents are xilene, benzene, toluene, heptane, cyclohexane and methyl tert-butyl ether.

Preferably, the reaction takes place in a mixture of heptane and toluene and, more preferably, the volume ratio between heptane and toluene is around 9:1 v/v.

The reaction temperature of the resolution process is comprised between room temperature and the reflux temperature of the solvent system used.

Preferably, the reaction is carried out at a temperature comprised between 30 and 60° C.

More preferably, reaction is carried out at a temperature around 50° C. followed by a controlled cooling phase in order to assist the isolation of the product in high diastereoisomeric excess.

A preferred embodiment of the invention comprises reacting the intermediate amide in heptane/toluene 9/1 v/v, at about 50° C. temperature in the presence of 10% sodium methoxide.

According to the invention, the synthetic scheme for the preparation of levetiracetam further comprises the hydrolysis reaction of the amide obtained by the dynamic process (hereinafter resolved amide) to give enantiomerically pure (−)-(S)-alpha-ethyl-2-oxo-1-pyrrolidineacetic acid and its transformation into the end product.

Therefore, it is another object of the present invention a process for the preparation of levetiracetam further comprising the hydrolysis of the resolved amide to give (−)-(S)-alpha-ethyl-2-oxo-1-pyaolidineacetic acid.

Generally, diastereoisomeric amide wherein chiral center in alpha position has the desired optical configuration is hydrolyzed to give said acid intermediate according to conventional methods.

In order to avoid an uncontrolled isomerization of the amide intermediate which may lead to loss in diastereoisomeric excess of the starting material and to prevent a racemization of reaction product (−)-(S)-alpha-ethyl-2-oxo-1-pyrrolidineacetic acid, the hydrolysis reaction is, preferably, carried out in acid conditions.

Suitable acids are strong inorganic acids such as hydrochloric acid, sulfuric acid or organic acids such as acetic acid, trifluoroacetic acid, p-toluensulfonic acid or alkyl-thiophenylsulfonic acid optionally supported on suitable polymeric or inorganic matrix.

The use of an organic acid is preferred since, under these reaction conditions, an improvement in chemoselectivity of hydrolysis process, i.e. a reduction of the by-product amount coming from pyrrolidine ring opening, up to complete lack of said by-product, is reached.

Moreover, between organic acids are particularly preferred strong organic acid such as p-toluensulphonic acid or alkyl-thiophenylsulfonic acid optionally supported on polymeric or inorganic matrix.

Hydrolysis reaction is carried out in the presence of an organic solvent.

Suitable organic solvents are aromatic hydrocarbons, lower alcohols and acetonitrile.

Preferred organic solvents are methanol and toluene.

Preferably, diastereoisomeric amide hydrolysis is carried out in toluene at reflux temperature.

Generally, conversion of (−)-(S)-alpha-ethyl-2-oxo-1-pyrrolidineacetic acid into the corresponding amide, levetiracetam, is carried out via activation of the carboxyl residue according to conventional techniques.

As reported in US '223, levetiracetam is prepared by the successive reaction of said acid with alkylhaloformate and ammonia.

Alternatively, carboxyl group may be activated as ester derivatives, for example, by reacting (−)-(S)-alpha-ethyl-2-oxo-1-pyrrolidineacetic acid with lower alcohols in the presence of an acid.

Subsequent ammonolysis reaction is preferably carried out in an aqueous medium.

Therefore, it is another object of the present invention a process for the preparation of levetiracetam further comprising the hydrolysis of the resolved amide to give (−)-(S)-alpha-ethyl-2-oxo-1-pyrrolidineacetic acid, activation of the carboxyl residue of said acid by esterification, ammonolysis of the resultant ester derivative and recovering the crude end-product.

In particular, considering that hydrolysis reaction can be carried out in acid conditions, in accordance with the above described teachings, it is evident to the man skilled in the art how activation of the carboxyl residue by exploiting said acid conditions entails significant procedural advantages.

From the practical point of view, it suffices that when hydrolysis completed a suitable amount of lower alcohol is added in the reaction mixture so that the correspondent pyrrolidine acetic ester derivative is obtained without isolating intermediate (−)-(S)-alpha-ethyl-2-oxo-1-pyrrolidineacetic acid.

In other words, hydrolysis and activation of the carboxyl residue are carried out by an acid catalyzed “one pot” hydrolysis-esterification reaction of the diastereoisomeric amide.

Preferably, the “one pot” hydrolysis-esterification reaction is carried out in the presence of p-toluensulfonic acid or alkyl-thiophenylsulfonic acid optionally supported on polymeric or inorganic matrix.

More preferably, styrene divinylbenzene polymer-bound p-toluensulfonic acid and silica-supported alkyl-thiophenylsulfonic acid are used.

Preferably, methyl alcohol, ethyl alcohol, isopropyl alcohol or n-butyl alcohol, methyl alcohol being more preferred, are added at hydrolysis completed.

In a preferred embodiment of the invention the “one pot” hydrolysis-esterification reaction is carried out in toluene at reflux temperature in the presence of p-toluensulphonic acid supported on polymeric matrix or alkyl-thiophenylsulfonic acid supported on silica followed by addition of methanol.

It is evident to the man skilled in the art the advantage deriving from using a heterogeneous acid reagent to carry out the “one pot” hydrolysis-esterification sequence. Indeed, an almost pure solution of the desired ester derivative in the reaction solvent is obtained by simply filtering the immobilized catalyst.

Preferably, ammonolysis reaction is carried out in the presence of water.

If necessary, crude levetiracetam may be purified by crystallization from an organic solvent or a mixture of organic solvents according to known methods.

A further aspect of the present invention refers to an intermediate compound of formula

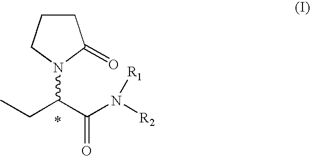

wherein

R₁ is hydrogen or a benzyl group;

R₂ is a 1-phenylethyl group optionally substituted on the phenyl ring by nitro or (C₁-C₄)-alkoxy; a 1-phenylpropyl group; a 3-pinylmethyl group;

or R₁ and R₂ taken together form a 5 or 6 membered saturated heterocycle containing from 1 to 3 heteroatoms selected among nitrogen, oxygen and sulfur, substituted by one or more (C₁-C₄)-alkyl group;

its stereoisomers, mixture thereof and acid addition salts.

The present invention comprises all stereoisomeric forms such as optical diastereoisomeric forms of the compounds of formula I and mixture thereof.

Preferred compounds are those wherein residue R₁ is a hydrogen atom.

In particular, the compounds:

(−)-(S)-alpha-ethyl-2-oxo-1-pyrrolidineacet-N-(+)-(R)-(1-phenylethyl)-amide;

(±)-(R,S)-alpha-ethyl-2-oxo-1-pyrrolidineacet-N-(+)-(R)-(1-phenylethyl)-amide;

(−)-(S)-alpha-ethyl-2-oxo-1-pyrrolidineacet-N-(−)-(S)-(1-phenylethyl)-amide;

(±)-(R,S)-alpha-ethyl-2-oxo-1-pyrrolidineacet-N-(−)-(S)-(1-phenylethyl)-amide;

(−)-(S)-alpha-ethyl-2-oxo-1-pyrrolidineacet-N-(+)-(R)-(1-phenylpropyl)-amide;

(±)-(R,S)-alpha-ethyl-2-oxo-1-pyrrolidineacet-N-(+)-(R)-(1-phenylpropyl)-amide;

(−)-(S)-alpha-ethyl-2-oxo-1-pyrrolidineacet-N-(−)-(S)-(1-phenylpropyl)-amide;

(±)-(R,S)-alpha-ethyl-2-oxo-1-pyrrolidineacet-N-(−)-(S)-(1-phenylpropyl)-amide;

(−)-(S)-alpha-ethyl-2-oxo-1-pyrrolidineacet-N-(+)-(R)-1-[(4-methoxyphenyl)-ethyl]-amide;

(±)-(R,S)-alpha-ethyl-2-oxo-1-pyrrolidineacet-N-(+)-(R)-1-[(4-methoxyphenyl)-ethyl]-amide;

(−)-(S)-alpha-ethyl-2-oxo-1-pyrrolidineacet-N-(−)-(S)-1-[(4-methoxyphenyl)-ethyl]-amide;

(±)-(R,S)-alpha-ethyl-2-oxo-1-pyrrolidineacet-N-(−)-(S)-1-[(4-methoxyphenyl)-ethyl]-amide;

(−)-(S)-alpha-ethyl-2-oxo-1-pyrrolidineacet-N-(+)-(R)-1-[(4-nitrophenyl)-ethyl]-amide;

(±)-(R,S)-alpha-ethyl-2-oxo-1-pyrrolidineacet-N-(+)-(R)-1-[(4-nitrophenyl)-ethyl]-amide;

(−)-(S)-alpha-ethyl-2-oxo-1-pyrrolidineacet-N-(−)-(S)-1-[(4-nitrophenyl)-ethyl]-amide;

(±)-(R,S)-alpha-ethyl-2-oxo-1-pyrrolidineacet-N-(−)-(S)-1-[(4-nitrophenyl)-ethyl]-amide;

(−)-(S)-alpha-ethyl-2-oxo-1-pyrrolidineacet-N-(+)-3-pinylmethyl-amide;

(±)-(R,S)-alpha-ethyl-2-oxo-1-pyrrolidineacet-N-(+)-3-pinylmethyl-amide;

(−)-(S)-alpha-ethyl-2-oxo-1-pyrrolidineacet-N-(−)-3-pinylmethyl-amide;

(±)-(R,S)-alpha-ethyl-2-oxo-1-pyrrolidineacet-N-(−)-3-pinylmethyl-amide;

(−)-(S)-alpha-ethyl-2-oxo-1-pyrrolidineacet-N-benzyl-N-(−)-(S)-(1-phenylethyl)-amide;

(±)-(R,S)-alpha-ethyl-2-oxo-1-pyrrolidineacet-N-benzyl-N-(−)-(S)-(1-phenylethyl)-amide;

(−)-(S)-alpha-ethyl-2-oxo-1-pyrrolidineacet-N-benzyl-N-(+)-(R)-(1-phenylethyl)-amide;

(±)-(R,S)-alpha-ethyl-2-oxo-1-pyrrolidineacet-N-benzyl-N-(+)-(R)-(1-phenylethyl)-amide;

(−)-(S)-alpha-ethyl-2-oxo-1-pyrrolidineacet-(−)-(R)-(3-methylpiperidin)-amide;

(±)-(R,S)-alpha-ethyl-2-oxo-1-pyrrolidineacet-(−)-(R)-(3-methylpiperidin)-amide;

(−)-(S)-alpha-ethyl-2-oxo-1-pyrrolidineacet-(+)-(S)-(3-methylpiperidin)-amide;

(±)-(R,S)-alpha-ethyl-2-oxo-1-pyrrolidineacet-(+)-(S)-(3-methylpiperidin)-amide;

(−)-(S)-alpha-ethyl-2-oxo-1-pyrrolidineacet-(−)-(R)-(2-methylpiperidin)-amide;

(±)-(R,S)-alpha-ethyl-2-oxo-1-pyrrolidineacet-(−)-(R)-(2-methylpiperidin)-amide;

(−)-(S)-alpha-ethyl-2-oxo-1-pyrrolidineacet-(+)-(S)-(2-methylpiperidin)-amide;

(±)-(R,S)-alpha-ethyl-2-oxo-1-pyrrolidineacet-(+)-(S)-(2-methylpiperidin)-amide;

(−)-(S)-alpha-ethyl-2-oxo-1-pyrrolidineacet-(2R,6R)-(2,6-dimethylpiperidin)-amide;

(±)-(R,S)-alpha-ethyl-2-oxo-1-pyrrolidineacet-(2R,6R)-(2,6-dimethylpiperidin)-amide;

(−)-(S)-alpha-ethyl-2-oxo-1-pyrrolidineacet-(−)-(R)-(2-methylpyrrolidin)-amide;

(±)-(R,S)-alpha-ethyl-2-oxo-1-pyrrolidineacet-(−)-(R)-(2-methylpyrrolidin)-amide;

(−)-(S)-alpha-ethyl-2-oxo-1-pyrrolidineacet-(+)-(S)-(2-methylpyrrolidin)-amide;

(±)-(R,S)-alpha-ethyl-2-oxo-1-pyrrolidineacet-(+)-(S)-(2-methylpyrrolidin)-amide;

(−)-(S)-alpha-ethyl-2-oxo-1-pyrrolidineacet-(2R,5S)-(2,5-dimethylpyrrolidin)-amide;

(±)-(R,S)-alpha-ethyl-2-oxo-1-pyrrolidineacet-(2R,5S)-(2,5-dimethylpyrrolidin)-amide;

are useful intermediates in the preparation of levetiracetam.

The compounds object of the present invention are prepared according to techniques known in the art, for example, by an amidation reaction of the corresponding acids or derivatives thereof.

The process of the present invention provides a resolution method very efficient from the industrial viewpoint which allows a good conversion into the desired optical isomer (diastereoisomeric excess around 96-99%) and prevents loss in yields of starting materials.

Thus, the process of the invention allows to obtain levetiracetam in high yields by a lower number of synthetic steps than conventional methods and, consequently, with reduced times and costs.

Moreover, a further advantage of the invention is represented by the opportunity of quantitatively recover the optically active amine when polymer bound p-toluensulfonic acid is used in the “one pot” hydrolysis-esterification step.

It is therefore readily apparent that the process of the present invention is advantageous with respect to those already described in the art.

A practical embodiment of the process object of the present invention comprises amidation reaction between a lower alkyl (±)-(R,S)-alpha-ethyl-2-oxo-1-pyrrolidine acetate and a suitable optical active amine, crystallization-induced dynamic resolution of the resultant diastereoisomeric acetamide from basic catalysis, hydrolysis of the resolved acetamide and conversion into levetiracetam.

An alternative practical embodiment of the present invention comprises amidation reaction between a lower alkyl (±)-(R,S)-alpha-ethyl-2-oxo-1-pyrrolidine acetate and a suitable optical active amine, dynamic resolution of the resultant diastereoisomeric acetamide from basic catalysis, one pot hydrolysis-esterification reaction of the resolved acetamide and conversion into levetiracetam.

A preferred practical embodiment of the present invention comprises reacting methyl (±)-(R,S)-alpha-ethyl-2-oxo-1-pyrrolidine acetate with (+)-(R)-(1-phenylethyl)-amine in toluene in the presence of a base such as sodium hydride or methoxide; crystallization-induced dynamic resolution of the resultant (±)-(R,S)-alpha-ethyl-2-oxo-1-pyrrolidineacet-N-(+)-(R)-(1-phenylethyl)-amide in heptane/toluene 9/1 v/v, at about 50° C. in the presence of 10% sodium methoxide; “one pot” hydrolysis-esterification reaction of the respective resolved (−)-(S)-alpha-ethyl-2-oxo-1-pyrrolidineacet-N-(+)-(R)-(1-phenylethyl)-amide by means of acid hydrolysis carried out in toluene at reflux temperature in the presence of p-toluensulphonic acid supported on polymeric matrix or alkyl-thiophenyl-sulfonic acid supported on silica followed by addition of methanol; and ammonolysis reaction in the presence of water.

It is to be understood that while the invention is described in conjunction of the preferred embodiments thereof, those skilled in the art are aware that other embodiments could be made without departing from the spirit of the invention.

For better illustrating the invention the following examples are now given.

### EXAMPLE 1

### (±)-(R,S)-alpha-ethyl-2-oxo-1-pyrrolidineacet-N-(+)-(R)-(1-phenylethyl)-amide

In a 100 ml reactor equipped with mechanical stirring, thermometer and bubble condenser, 13.4 g of (±)-(R,S)-alpha-ethyl-2-oxo-1-pyrrolidineacetic acid methyl ester (71.6 mmol), 8.8 g of (+)-(R)-(1-phenylethyl)-amine (72 5 mmol) and 45 ml of tetrahydrofuran were charged. 3.4 g of NaH (60% dispersion in mineral oil, 85.6 mmol) was added in small portions under nitrogen atmosphere. Reaction mixture was maintained at room temperature for about 2 h. Then, it was heated up to 35° C. and kept under stirring overnight. Reaction was controlled by TLC (Rf=0.5, AcOEt/silica gel).

At reaction completed, one night at 35° C. temperature, reaction mixture was cooled to room temperature and 30 ml of water was slowly charged. It was transferred into a separatory funnel and was diluted with 30 ml of water and 80 ml of dichloromethane.

Phases were separated and the aqueous one was washed with 50 ml of dichloromethane. Collected organic phases were washed with an aqueous acid solution, dried on Na₂SO₄, filtered and concentrated under vacuum.

19.5 g of an oil residue was obtained which slowly solidified. Solid was suspended in 20 ml of a hexane/dichloromethane 9/1 v/v mixture. It was then filtered, washed with 10 ml of the same solvent mixture and dried at 40° C. to give 12.1 g of the title compound (44.1 mmol, 61.6% yield) as dry solid.

¹H NMR (400.13 MHz, CDCl₃, 25° C.): δ (ppm, TMS) 7.35-7.19 (10H, m), 6.49 (2H, br s), 5.09-5.00 (2H, m), 4.41 (1H, dd, J=8.3, 7.4 Hz), 4.36 (1H, dd, J=8.6, 7.1 Hz), 3.49 (1H, ddd, J=9.8, 7.7, 6.6 Hz), 3.41 (1H, ddd, J=9.8, 7.7, 6.2 Hz), 3.30 (1H, ddd, J=9.6, 8.3, 5.5 Hz), 3.13 (1H, ddd, 9.7, 8.5, 6.1 Hz), 2.47-2.38 (2H, m), 2.41 (1H, ddd, J=17.0, 9.6, 6.3 Hz), 2.26 (1H, ddd, 17.0, 9.5, 6.6 Hz), 2.10-1.98 (2H, m), 2.01-1.89 (1H, m), 1.99-1.88 (1H, m), 1.98-1.85 (1H, m), 1.88-1.78 (1H, m), 1.75-1.62 (1H, m), 1.72-1.59 (1H, m), 1.45 (3H, d, J=7.1 Hz), 1.44 (3H, d, J=7.1 Hz), 0.90 (3H, t, J=7.4 Hz), 0.86 (3H, t, J=7.4 Hz). ¹³C NMR (100.62 MHz, CDCl₃, 25° C.): δ (ppm, TMS) 176.05 (CO), 176.00 (CO), 169.08 (CO), 168.81 (CO), 143.59 (C_{quat}), 143.02 (C_{quat}), 128.66 (2×CH), 128.55 (2×CH), 127.33 (CH), 127.19 (CH), 126.05 (2×CH), 125.80 (2×CH), 56.98 (CH), 56.61 (CH), 48.90 (CH), 48.84 (CH), 44.08 (CH₂), 43.71 (CH₂), 31.19 (CH₂), 31.07 (CH₂), 22.08 (CH₃), 22.04 (CH₃), 21.21 (CH₂), 20.68 (CH₂), 18.28 (CH₂), 18.08 (CH₂), 10.50 (CH₃), 10.45 (CH₃).

### EXAMPLE 2

### (±)-(R,S)-alpha-ethyl-2-oxo-1-pyrrolidineacet-N-(+)-(R)-(1-phenylethyl)-amide (Alternative 1)

In a 500 ml reactor equipped with mechanical stirring, thermometer and condenser, 24.2 g of (+)-(R)-(1-phenylethyl)-amine (199.51 mmol) and 40 ml of toluene were charged. By keeping the reaction mixture at 0° C. temperature under nitrogen atmosphere, 9.5 g of NaH (60% mineral oil suspension, 237.50 mmol) was added in small portions. At the same temperature, 190.0 g of a toluene solution of (±)-(R,S)-alpha-ethyl-2-oxo-1-pyrrolidineacetic acid methyl ester (19.28% equal to 36.63 g, 197.77 mmol) was charged. Reaction mixture was then heated up to 35° C. and maintained in that condition till complete disappearing of methyl ester reagent (about 14 h; checked by HPLC).

At reaction completed, reaction mixture was cooled and when room temperature was reached, 100 ml of water was slowly charged. Aqueous phases were separated and extracted with toluene (2×75 ml). Collected organic phases were treated with acid water till neuter pH. Solvent was evaporated and residue was suspended in about 100 ml of heptane for about 30 minutes. Product was isolated by filtration and dried in oven at 40° C. temperature under vacuum overnight to give 45.2 g of the title compound (164.54 mmol, 83.2% yield, d.e. 0.0%) as white dusty solid.

### EXAMPLE 3

### (±)-(R,S)-alpha-ethyl-2-oxo-1-pyrrolidineacet-N-(+)-(R)-(1-phenylethyl)-amide (Alternative 2)

In a 500 ml reactor equipped with mechanical stirring, thermometer and Dean-Stark distiller, 24.2 g of (+)-(R)-(1-phenylethyl)-amine (199.51 mmol) and 40 ml of toluene were charged. By keeping the reaction mixture at 0° C. temperature, 42.7 g of sodium methoxide (30% solution in methanol, 237.14 mmol) was added under nitrogen atmosphere. At the same temperature, 190.0 g of a toluene solution of (±)-(R,S)-alpha-ethyl-2-oxo-1-pyrrolidineacetic acid methyl ester (19.28% equal to 36.63 g, 197.77 mmol) was charged. Reaction mixture was then heated up to 65-70° C. and maintained in that condition till complete disappearing of methyl ester reagent (about 4 h; checked by HPLC). After a work-up carried out according to the procedure described in example 2, 40.2 g of the title compound (146.53 mmol, 74.1% yield, d.e. 0.0%) as white dusty solid was obtained.

### EXAMPLE 4

### (−)-(S)-alpha-ethyl-2-oxo-1-pyrrolidineacet-N-(+)-(R)-(1-phenylethyl)-amide

In a 25 ml reactor equipped with a bubble condenser and mechanical stirring, 1.5 g of (±)-(R,S)-alpha-ethyl-2-oxo-1-pyrrolidineacet-N-(+)-(R)-(1-phenylethyl)-amide (5.47 mmol) and 15 ml of a n-heptane/toluene 9/1 v/v mixture were charged under nitrogen atmosphere. Reaction mixture was heated up to 50° C. temperature and about 100 mg of sodium methoxide (30% solution in methanol, 0.55 mmol) was charged. Quickly, suspension dissolved and an oil was formed.

Reaction mixture was cooled to 40° C. temperature, kept under stirring overnight and resulting suspension was cooled to 20° C. in about 4 h. To the suspension was added 50 mg of acetic acid and then it was filtered. So obtained solid was washed with n-heptane (1×5 ml) and was dried under vacuum at 50° C. temperature overnight to give the title compound (1.1 g, 4.0 mmol, 73.3% yield, d.e.=91.8%) as white solid.

¹H NMR (400.13 MHz, CDCl₃, 25° C.): δ (ppm, TMS) 7.33-7.18 (5H, m), 6.54 (1H, br d, J=7.4 Hz), 5.04 (1H, dt, J=7.4, 7.1 Hz), 4.41 (1H, dd, J=8.3, 7.4 Hz), 3.30 (1H, ddd, J=9.6, 8.3, 5.5 Hz), 3.13 (1H, ddd, 9.7, 8.5, 6.1 Hz), 2.41 (1H, ddd, J=17.0, 9.6, 6.3 Hz), 2.26 (1H, ddd, 17.0, 9.5, 6.6 Hz), 2.01-1.89 (1H, m), 1.99-1.88 (1H, m), 1.88-1.78 (1H, m), 1.72-1.59 (1H, m), 1.45 (3H, d, J=7.1 Hz), 0.90 (3H, t, J=7.4 Hz). ¹³C NMR (100.62 MHz, CDCl₃, 25° C.): δ (ppm, TMS) 176.05 (CO), 168.81 (CO), 143.59 (C_{quat}), 128.55 (2×CH), 127.19 (CH), 125.80 (2×CH), 56.61 (CH), 48.84 (CH), 43.71 (CH₂), 31.07 (CH₂), 22.08 (CH₃), 20.68 (CH₂), 18.08 (CH₂), 10.45 (CH₃).

### EXAMPLE 5

### (−)-(S)-alpha-ethyl-2-oxo-1-pyrrolidineacetic acid

In a 25 ml flask equipped with mechanical stirring and bubble condenser, 1.0 g of (−)-(S)-alpha-ethyl-2-oxo-1-pyrrolidineacet-N-(+)-(R)-(1-phenylethyl)-amide (3.65 mmol, d.e.=98%), 7.3 g of p-toluensulfonic acid supported by polymeric matrix (30.00-60.00 mesh, 2.0-3.0 mmol/g), 0.263 ml of water (14.60 mmol) and 14.5 ml of toluene were charged under nitrogen atmosphere.

Reaction mixture was heated up to 110° C. temperature by oil bath and maintained at reflux temperature up to complete disappearing of starting material (about 6 h; checked by HPLC). Reaction checks were made by taking both a portion of liquid phase and an amount of resin; mixture was filtered, washed with about 2 ml of an ammonia solution (7.0 M in MeOH) and solvent was eliminated under vacuum.

At complete conversion, reaction mixture was filtered on gootch, resin was washed with aqueous NaOH 1M (2×15 ml) and 10 ml of toluene. Phases were separated and toluene solution was washed with 15 ml of soda 1 M in water up to pH value around 10-12. So obtained aqueous basic phase was further washed with 20 ml of toluene and then acidified with 3% aqueous HCl up to pH value around 1. Aqueous acid solution was extracted with dichloromethane (5×50 ml). Collected organic phases were dried on Na₂SO₄, and concentrated under vacuum up to a residue was formed. So obtained white solid was dried under vacuum at 25° C. temperature overnight to give 304.0 mg of the title compound (1.78 mmol, 48.7% yield, e.e.=91.9%).

### EXAMPLE 6

### (−)-(S)-alpha-ethyl-2-oxo-1-pyrrolidineacetamide (Levetiracetam)

In a 25 ml flask equipped with thermometer, mechanical stirring and bubble condenser, 3.344 g of (−)-(S)-alpha-ethyl-2-oxo-1-pyrrolidineacetic acid (19.58 mmol, e.e.=95.0%), 0.11 ml of concentrated sulfuric acid (95.6% m/m, 1.97 mmol) and 17 ml of methanol were charged under nitrogen atmosphere at room temperature.

Reaction mixture was heated up to 65° C. temperature by oil bath and maintained at reflux temperature up to complete disappearing of starting material (about 2.5 h; checked by TLC, Rf=0.58 CH₂Cl₂:MeOH:AcOH 80:20:1/silica gel). Reaction mixture was concentrated under vacuum up to a residue was formed then water (2.0 ml) was added. In a 25 ml flask equipped with magnetic stirring and condenser, 7.5 ml of 30% aqueous ammonia solution was charged and cooled to 0° C. temperature and, keeping under stirring, the aqueous solution of crude (−)-(S)-alpha-ethyl-2-oxo-1-pyrrolidineacetic acid methyl ester was charged dropwise. When addition was completed, reaction mixture was thermostabilized at 20° C. and said conditions were maintained overnight.

At complete conversion (about 10 h) excess of ammonia was eliminated by vacuum evaporator. Reaction mixture was extracted with dichloromethane (2×3.5 ml), transferred into a continuous liquid-liquid extractor and then refluxed with 7 ml of dichloromethane for 6 hours. Collected organic phases were concentrated under vacuum up to a residue was formed. 2.666 g of a yellow solid was obtained which was suspended in 15.0 ml of acetone. Reaction mixture was heated up to 60° C. temperature so that complete dissolution of the solid was reached. Then, mixture was slowly cooled. White solid was isolated by filtration, washed with mother liquors and then with 3 ml of cold acetone and, finally, dried in oven under vacuum at 40° C. temperature for 4 hours to give 2.259 g of levetiracetam (13.274 mmol, 67.8% yield, e.e. 99.9%).

### EXAMPLE 7

### (−)-(S)-alpha-ethyl-2-oxo-1-pyrrolidineacetic acid methyl ester

In a 250 ml reactor equipped with mechanical stirring, thermometer and condenser, 2.5 g of (−)-(S)-alpha-ethyl-2-oxo-1-pyrrolidineacet-N-(+)-(R)-(1-phenylethyl)-amide (9.112 mmol, d.e.=99.3%), 24.85 g (6 eq.) of p-toluensulfonic acid supported by polymeric matrix (30.00-60.00 mesh, 2.2 mmol/g) and 75 ml of toluene were charged. To the reaction mixture was added 0.660 ml (36.64 mmol) of water under stirring and mixture was heated up to reflux temperature. Reaction was monitored by HPLC and at complete conversion of starting material (about 6 h), mixture was cooled to 60° C. temperature and 75 ml of methanol added. Reaction mixture was maintained at that temperature for 3 h up to complete formation of (−)-(S)-alpha-ethyl-2-oxo-1-pyrrolidineacetic acid methyl ester. Reaction mixture was permitted to cool and then it was filtered on gootch in order to separate the product from the resin. Resin was washed with methanol (2×75 ml) and organic phases were collected to give 365.1 g of a 0.462% organic solution of (−)-(S)-alpha-ethyl-2-oxo-1-pyrrolidineacetic acid methyl ester (1.69 g, 9.110 mmol, 100.0% yield) which was used in the following synthetic step.

In order to recover (+)-(R)-(1-phenylethyl)-amine, resin was treated with 100 ml of 30% aqueous ammonia solution, 100 ml of methanol, 100 ml of 30% aqueous soda and again with 100 ml of methanol. Resin was then regenerated by washing with HCl 6 M (100 ml) and water up to neuter pH of the eluted phase. Finally, resin was washed with 100 ml of methanol and dried in oven at 50° C. temperature under vacuum overnight.

### EXAMPLE 8

### (−)-(S)-alpha-ethyl-2-oxo-1-pyrrolidineacetamide (Levetiracetam) (Alternative 1)

365.1 g of the solution of (−)-(S)-alpha-ethyl-2-oxo-1-pyrrolidineacetic acid methyl ester (0.462%, 1.69 g, 9.110 mmol) obtained in Example 7 was charged in a flask and concentrated up to a residue was formed. 2.482 g of a brown oil was obtained.

Residue was charged in a 10 ml flask equipped with magnetic stirring and condenser.

Reaction mixture was cooled to 0° C. temperature and, keeping under stirring, 0.8 ml of water and 3.2 ml of 30% aqueous ammonia solution were charged dropwise in about 10 minutes. When addition was completed, reaction mixture was thermostabilized at 20° C. and said conditions were maintained overnight.

At complete conversion (about 14 h) excess of ammonia was eliminated by vacuum evaporator. Reaction mixture was then extracted with dichloromethane (10×5 ml). Collected organic phases were dried on Na₂SO₄, and concentrated under vacuum up to a residue was formed. 1.999 g of a yellow solid was obtained which was suspended in 5 ml of acetone. Reaction mixture was heated up to 60° C. temperature so that complete dissolution of the solid was reached. Then, mixture was slowly cooled. White solid was isolated by filtration, washed with mother liquors and then with 1 ml of cold acetone and, finally, dried in oven under vacuum at 25° C. temperature for 1 night to give 0.965 g of levetiracetam (5.669 mmol, 62.2% yield, e.e. 94.2%).

### EXAMPLE 9

### (−)-(S)-alpha-ethyl-2-oxo-1-pyrrolidineacetamide (Levetiracetam) (Alternative 2)

In a 50 ml reactor equipped with mechanical stirring, thermometer and condenser, 0.275 g of (−)-(S)-alpha-ethyl-2-oxo-1-pyrrolidineacet-N-(+)-(R)-(1-phenylethyl)-amide (1.0 mmol, d.e.=99.3%), 10.0 g of ethyl-thiophenyl-sulfonic acid supported on silica (0.6 mmol/g, supplied by Phosphonics®) and 15 ml of toluene were charged.

To the reaction mixture was added 0.075 ml (4.0 mmol) of water under stirring and mixture was heated up to reflux temperature. Reaction is monitored by HPLC and at complete conversion of starting material (about 5 h), reaction mixture was cooled to 60° C. temperature and 10 ml of methanol added. Reaction mixture was maintained at that temperature for 3 h up to complete formation of (−)-(S)-alpha-ethyl-2-oxo-1-pyrrolidineacetic acid methyl ester. Reaction mixture was permitted to cool and then worked up according to the procedure described in example 7. 57.9 g of a 0.280% organic solution of (−)-(S)-alpha-ethyl-2-oxo-1-pyaolidineacetic acid methyl ester (0.162 g, 0.875 mmol, 87.5% yield) was thus obtained. Such solution was charged in a flask and concentrated up to a residue was formed. 0.486 g of a brown oil was obtained. Residue was charged in a 5 ml flask equipped with magnetic stirring and condenser. Reaction mixture was cooled to 0° C. temperature and, keeping under stirring, 1.5 ml of 30% aqueous ammonia solution were charged dropwise. When addition was completed, reaction mixture was thermostabilized at 20° C. and said conditions were maintained overnight.

At complete conversion (about 15 h) excess of ammonia was eliminated by vacuum evaporator. Reaction mixture was then extracted with dichloromethane as described in example 8. Recrystallization of the crude product from refluxing acetone afforded 0.076 g of levetiracetam (0.447 mmol, 44.6% yield compared to the starting amide, e.e. 99.9%).

1. A process for the preparation of levetiracetam which comprises a crystallization-induced dynamic resolution of a diastereoisomeric mixture of an (±)-alpha-ethyl-2-oxo-1-pyrrolidine acetic amide of formula 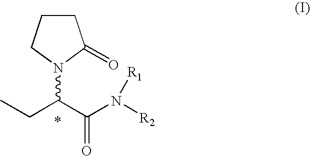
wherein
R1 is hydrogen or a benzyl group;
R2 is a 1-phenylethyl group optionally substituted on the phenyl ring by nitro or (C1-C4)-alkoxy; a 1-phenylpropyl group; a 1-naphtylethyl group; a 3-pinylmethyl group;
or R1 and R2 taken together form a 5 or 6 membered saturated heterocycle containing from 1 to 3 heteroatoms selected among nitrogen, oxygen and sulfur, substituted by one or more (C1-C4)-alkyl group;
from basic catalysis.

2. A process according to claim 1 wherein R1 is hydrogen.

3. A process according to claim 2 wherein the acetic amide of formula I is (±)-(R,S)-alpha-ethyl-2-oxo-1-pyrrolidineacet-N-(+)-(R)-(1-phenylethyl)-amide.

4. A process according to claim 1 wherein dynamic resolution is carried out in the presence of a catalytic amount of an organic base.

5. A process according to claim 4 wherein the organic base is selected from 1,4-diazabicyclo[2.2.2]octane, 1,8-diazabicyclo[5.4.0]undec-7-ene, 1,5,7-triazabicyclo[4.4.0]dec-5-ene and an alkali metal alkoxide.

6. A process according to claim 5 wherein the organic base is a (C1-C4)-alkali metal alkoxide.

7. A process according to claim 6 wherein the organic base is sodium methoxide.

8. A process according to claim 1 wherein catalytic amount of base is comprised between 5% and 15%.

9. A process according to claim 8 wherein catalytic amount of base is around 10%.

10. A process according to claim 1 further comprising the hydrolysis of the resolved amide to give (−)-(S)-alpha-ethyl-2-oxo-1-pyrrolidineacetic acid.

11. A process according to claim 10 wherein hydrolysis is carried out in acid conditions.

12. A process according to claim 11 wherein hydrolysis is carried out in the presence of p-toluensulfonic acid or alkyl-thiophenylsulfonic acid optionally supported on polymeric or inorganic matrix.

13. A process according to claim 1 further comprising the hydrolysis of the resolved amide to give (−)-(S)-alphaethyl-2-oxo-1-pyrrolidineacetic acid, activation of the carboxyl residue of said acid by esterification, ammonolysis of the resultant ester derivative and recovering the crude end-product.

14. A process according to claim 13 wherein hydrolysis and activation of the carboxyl residue are carried out by an acid catalyzed “one pot” hydrolysis-esterification reaction.

15. A process according to claim 14 wherein “one pot” hydrolysis-esterification reaction is carried out in the presence of styrene divinylbenzene polymer-bound p-toluensulfonic acid or silica supported alkyl-thiophenylsulfonic acid.

16. A process according to claim 14 wherein methyl alcohol, ethyl alcohol, isopropyl alcohol or n-butyl alcohol are added at hydrolysis completed.

17. A process according to claim 16 wherein methyl alcohol is added.

18. A process according to claim 13 wherein ammonolysis reaction is carried out in the presence of water.

19. A compound of formula 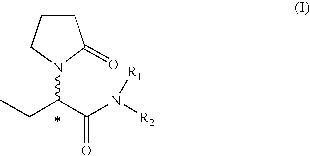
wherein
R1 is hydrogen or a benzyl group;
R2 is a 1-phenylethyl group optionally substituted on the phenyl ring by nitro or (C1-C4)-alkoxy; a 1-phenylpropyl group; a 3-pinylmethyl group;
or R1 and R2 taken together form a 5 or 6 membered saturated heterocycle containing from 1 to 3 heteroatoms selected among nitrogen, oxygen and sulfur, substituted by one or more (C1-C4)-alkyl group;
its stereoisomers, mixture thereof and acid addition salts.

20. A compound according to claim 19 wherein R1 is hydrogen.

21. A compound according to claim 20, wherein the acetic amide of formula I is (−)-(S)-alpha-ethyl-2-oxo-1-pyrrolidineacet-N-(+)-(R)-(1-phenylethyl)-amide.

22. A compound according to claim 20, wherein the acetic amide of formula I is (±)-(R,S)-alpha-ethyl-2-oxo-1-pyrrolidineacet-N-(+)-(R)-(1-phenylethyl)-amide.

23. A process according to claim 3 wherein catalytic amount of base is comprised between 5% and 15%.

24. A process according to claim 4 wherein catalytic amount of base is comprised between 5% and 15%.

25. A process according to claim 5 wherein catalytic amount of base is comprised between 5% and 15%.

26. A process according to claim 6 wherein catalytic amount of base is comprised between 5% and 15%.

27. A process according to claim 7 wherein catalytic amount of base is comprised between 5% and 15%.
